(19) United States
(12) Patent Application Publication
(10) Pub. No.: US 2025/0010374 A1
(43) Pub. Date: Jan. 9, 2025

(54) METHOD AND DEVICE FOR CHARACTERIZING A METAL POWDER

(71) Applicant: COMMISSARIAT A L’ENERGIE ATOMIQUE ET AUX ENERGIES ALTERNATIVES, PARIS (FR)

(72) Inventors: Natalia SERGEEVA-CHOLLET, Gif-sur-Yvette (FR); Romain SADDOUD, Gif-sur-Yvette (FR); Kévyn PERLIN, Gif-sur-Yvette (FR); Jean-Paul GARANDET, Gif-sur-Yvette (FR)

(21) Appl. No.: 18/764,209

(22) Filed: Jul. 4, 2024

(30) Foreign Application Priority Data
Jul. 5, 2023 (FR) 2307173

(51) Int. Cl. B22F 10/80; B33Y 50/00; G01N 27/90

(52) U.S. Cl. CPC B22F 10/80; G01N 27/90; B22F 2301/35; B33Y 50/00

(57) ABSTRACT

A device is provided for characterizing a residual powder remaining after an operation of additive manufacturing by a powder bed fusion process. The device includes: a powder receptacle for receiving a sample of residual powder, being calibrated in dimensions to contain the sample and comprising an upper lid composed of a covering film and a sealing lid; an eddy current emitting/receiving apparatus having a coil with a diameter dimensioned in accordance with the powder receptacle, and configured to emit to the surface of the powder sample, through the covering film, electromagnetic signals within a range of predefined working frequencies; and an acquisition and control system, configured to: measure, for each working frequency, impedance values across the terminals of a receiving coil of the eddy current emitting/receiving apparatus; and evaluate the quality of the remaining powder according to the impedance values measured for the powder sample.

### CROSS-REFERENCE TO RELATED APPLICATION

This application claims priority to foreign French patent application No. FR 2307173, filed on Jul. 5, 2023, the disclosure of which is incorporated by reference in its entirety.

### FIELD OF THE INVENTION

The present invention relates to the field of the characterization of metal powder, and is more particularly directed to the monitoring via eddy currents of the quality of a residual austenitic steel powder after an additive manufacturing operation.

### BACKGROUND

According to the standard NF E 67-001, the term Additive Manufacturing (AM) denotes “all the processes for manufacturing, layer by layer by the addition of material, a physical object starting from a digital object”. This term encompasses dozens of manufacturing technologies, classified into seven process categories according to the standard NF ISO 17296-2 Jun. 2015.

Powder bed fusion (PBF) processes, in which the powder is locally melted and resolidified, share the common feature of performing the partial or total melting of a powder generally originating from metal, ceramic or plastics materials.

PBF processes differ according to the nature of the energy source employed to bring about the melting, where this may for example be a laser (the process then being called L-PBF for laser powder bed fusion) or an electron beam (the process then being called EB-PBF for electron beam powder bed fusion).

In the additive manufacturing of metal parts, in particular by PBF, L-PBF and EB-PBF processes, the amount of powder used during the manufacturing of a part rarely exceeds 30% of the total volume of powder employed for the entire manufacturing process.

In some cases, in order not to take any risks in a subsequent operation, the non-melted powder recovered is directly disposed of or is subjected to a lengthy recycling process (melting and atomization) in order to use only fresh powder and ensure the quality of future manufactured parts. This leads to an increase in the cost of the part.

In view of the amount of powder remaining after an operation of manufacturing a part, it is thus of interest, at least for cost reasons, to be able to reuse the remaining metal powder in future manufacturing operations.

However, the reuse of the powder can be envisioned only for a residual powder having a quality which is monitored effectively. This is because, during the manufacturing process, some of the powder has been exposed to elevated temperatures, which may have changed the properties of the powder.

In particular, in L-PBF manufacturing, certain particles in the vicinity of the solidified beads may be entrained by the gas flow and be melted under the effect of the laser (these are hot ejecta).

In the case of austenitic steel powders (which is a specific type of stainless steel alloy containing austenite), depending on their cooling kinetics, these particles may resolidify in ferrite form. And, because of their magnetic properties, the ferritic particles present a problem in controlling the spread of the powder for the additive manufacturing operations.

Consequently, direct reuse of such a used powder could have the harmful effects of degrading the properties of a part that would be manufactured with it.

According to the standard ASTM F3184 (“Standard Specification for Additive Manufacturing Stainless Steel Alloy (UNS S31603) with Powder Bed Fusion”), the reuse of powder is permitted an unlimited number of times provided that the quality of the final manufactured part remains constant.

Also, it is essential to monitor the quality of a residual powder after use in additive manufacturing with a view to reusing all or some of the remaining powder for a new manufacture of a part.

An article by T. Delacroix, et al. “Influence of powder recycling on 316 L stainless steel feedstocks and printed parts in laser powder bed fusion” (Addit. Manuf. 2021, 50, 102553) studies the presence of ferrite in various types of powder: fresh powder or powder recycled up to 15 times. The reference method for finely characterizing the presence of ferrite is electron backscatter diffraction (EBSD).

This method can provide very precise information on the composition of the powder and the crystal orientation of the particles. However, it requires specific preparation of the samples and needs a significant amount of time for the measurements and analyses. Moreover, it only allows processing of very limited amounts of material, and is thus not suitable for an industrial environment.

On a slightly larger scale, X-ray diffraction (XRD) enables quantification of the observed phases with less of a need for sample preparation, but the amounts of material investigated remain limited and the XRD technique is not suitable for an industrial environment either.

There is therefore a need for an appropriate solution for characterizing a residual metal powder after an operation of manufacturing a part by additive manufacturing, and in particular for determining the presence of ferrite particles in such a powder.

The present invention addresses this need.

### SUMMARY

A subject of the present invention relates to a method and a device for characterizing a metal powder.

An aim of the present invention is to overcome the abovementioned drawbacks of the known approaches by proposing a method, and an associated device, for determining the presence of ferrite particles in a residual metal powder after an additive manufacturing operation according to a powder bed fusion process.

The general principle of the invention consists of monitoring, by eddy current measurements, the appearance of ferritic particles in a stainless steel powder recovered after an additive manufacturing operation according to a powder bed fusion process.

The device of the invention combines the use of a receptacle for receiving the powder to be characterized, the receptacle having a volume determined according to the characteristics of the additive manufacturing application, with the use of an eddy current measurement sensor.

Advantageously, the receptacle is designed to take measurements of a sufficiently representative powder sample, making it possible to evaluate the quality of the powder recovered.

Advantageously, the eddy current sensor according to the invention is optimized to be sensitive to changes in the electromagnetic properties of a powder in the presence of ferrite particles.

The invention will find advantageous applications in numerous technical fields such as the aeronautical, space, automotive and nuclear industries, to cite but a few examples.

In order to obtain the desired results, a device for characterizing a powder according to the independent device claim is proposed.

The device comprises:
a powder receptacle configured to receive a sample of residual powder remaining after an operation of additive manufacturing by a powder bed fusion process, the receptacle being calibrated in terms of its dimensions to contain said sample and comprising an upper lid composed of a covering film and a sealing lid;
an eddy current emitting/receiving apparatus having a coil with a diameter dimensioned in accordance with the dimensions of the powder receptacle, and configured to emit to the surface of the powder sample, through the covering film, electromagnetic signals within a range of predefined working frequencies; and
an acquisition and control system, configured to:
   measure, for each working frequency, impedance values across the terminals of a receiving coil of the eddy current emitting/receiving apparatus; and
   evaluate the quality of the remaining powder according to the impedance values measured for the powder sample.

According to some alternative or combined embodiments:
The powder receptacle comprises a main body comprising at least one powder receiving chamber, and a bearing block for pressing the powder, the powder receiving chamber being dimensioned to receive a powder sample representative of the powder remaining after manufacture.
The powder receptacle comprises an air evacuation device configured to suck out the air contained in the powder receptacle.
The receptacle is made from a range of non-metallic materials, including in particular Delrin (polyoxymethylene POM), Plexiglas (polymethyl methacrylate), acrylic (polyacrylonitrile PAN), Mylar (PET polyester film), rubber.
The eddy current emitting/receiving apparatus comprises a sensor composed of a coil (which may be the same for the excitation and reception functions) without amplification in absolute mode, the diameter of the coil being dimensioned in accordance with the dimensions of the powder receptacle.
The eddy current emitting/receiving apparatus comprises a sensor composed of a coil (which may be the same for the excitation and reception functions) of which the number of layers and the number of turns is predefined in order to ensure that the resonant frequency of the coil is higher than the range of working frequencies of the sensor.
The acquisition and control system comprises an impedance meter for measuring the impedance values.

The invention is also directed to a method for characterizing a residual powder remaining after an operation of additive manufacturing by a powder bed fusion process according to the independent method claim. The method is implemented for a sample of residual powder contained in a powder receptacle calibrated in terms of its dimensions to contain said sample and comprising an upper lid composed of a covering film and a sealing lid. The method comprises the steps of:
activating, via an eddy current EC sensor having a coil with a diameter dimensioned in accordance with the dimensions of the powder receptacle, the emission of electromagnetic signals, within a range of predefined working frequencies, to the surface of said powder sample, through the covering film;
measuring, for each working frequency, the impedance across the terminals of the receiving coil of the EC sensor; and
evaluating the quality of the remaining powder according to the impedance values measured for the powder sample.

According to some alternative or combined embodiments:
The activation step of the EC sensor is carried out within a range of working frequencies between 2 MHz and 10 MHz.
The activation step of the EC sensor is carried out within a range of working frequencies between 4 MHz and 5 MHz.
The step of evaluating the quality of the remaining powder comprises a step of comparing the impedance measurements with predefined impedance values and of quantifying the presence of ferrite particles in the powder sample according to the differences between the measured values and the predefined values.
The step of evaluating the quality of the remaining powder comprises a step of defining an acceptable ratio of ferrite particles present in the powder sample for retaining the remaining powder for a subsequent additive manufacture or not.
The characterization of a powder consists of determining the presence of ferrite particles in a sample of 316 L-type austenitic powder.

### BRIEF DESCRIPTION OF THE DRAWINGS

Various aspects and advantages of the invention will become apparent on the basis of the description of a preferred but nonlimiting mode of implementation of the invention, with reference to the figures below:

FIG. 1 is a schematic representation of a powder receptacle for carrying out measurements according to the principles of the invention in one embodiment.

FIG. 2 is an exploded view of the powder receptacle of FIG. 1.

FIG. 3 is a schematic representation of the device for characterizing a powder according to one embodiment of the invention.

FIG. 4 illustrates the general steps of the method for determining the presence of ferrite particles in a powder according to the invention.

FIG. 5a and FIG. 5b respectively illustrate compared measurements of the reactance and of the resistance of a fresh powder and of a recycled powder.

### DETAILED DESCRIPTION

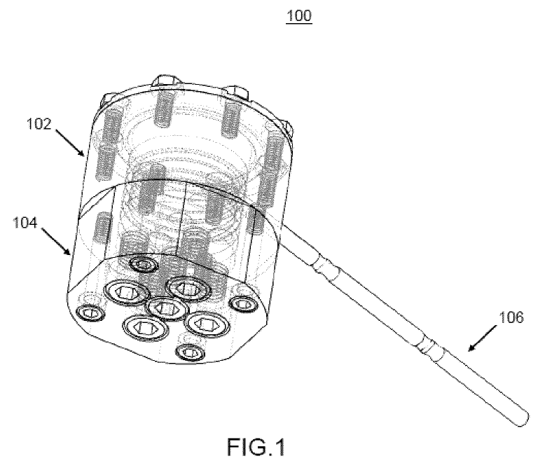

FIG. 1 schematically illustrates an embodiment of a receptacle according to the invention, intended to receive a powder sample in order to carry out characterization measurements of the powder according to the principles of the invention.

The receptacle 100 is made so as to carry out measurements on a mass of powder representative of the powder existing at the end of an additive manufacturing operation (this powder is also denoted as being a residual powder).

The receptacle 100 is principally composed of two parts: a main body 102 comprising at least one powder receiving chamber, and a bearing block 104 for pressing the powder. The powder is pressed against the upper part of the receptacle (receiving an eddy current sensor) in order to avoid having air between the sensor and the powder and thus to avoid distorting the result of the measurement.

The receptacle 100 is also equipped with an air evacuation device 106.

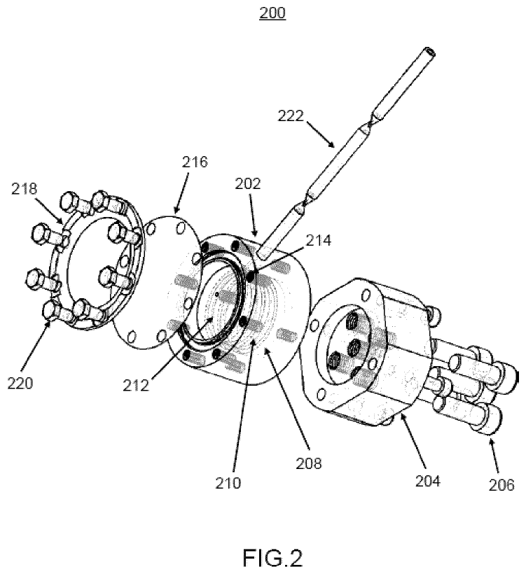

Other components for the assembly of the structure and the operation are described in detail with reference to FIG. 2.

Advantageously, a receptacle is developed and dimensioned for a given application. In particular, the diameter of the powder receiving chamber is calculated for receiving a mass of powder representative of the powder recovered after an additive manufacturing operation.

The volume of powder that allows measurements to be taken according to the principles of the invention is typically of the order of around one hundred grams. By way of nonlimiting example, for a mass of powder of between 50 and 250 grams, the diameter of the chamber of the receptacle may be adjusted between 30 and 75 millimeters. A person skilled in the art will be able to adapt sizes of the receptacles to other powder volumes while retaining the measurement principles of the invention.

FIG. 2 is an exploded view of the receptacle of FIG. 1 in one embodiment.

The receptacle 200 is composed of a main body 202 which is preferentially made from a transparent material in order to visualize the powder.

In a particular embodiment, the main body is made from Plexiglas.

The main body 202 comes to bear against a bearing block 204. In one embodiment, the main body is screwed onto the bearing block by means of a set of screws 206. In one particular embodiment, four stainless steel M10×35 mm screws enable the two blocks to be sealed together.

The main body 202 comprises a displaceable internal piston 208 with O rings 210 for sealing (two in FIG. 2), in order to be able to adjust the volume of the powder 212 inside the receptacle. The powder volume is adjusted using the bearing block 204 with the screws 206 at the back of the receptacle.

In one particular embodiment, the piston is a piston made from Delrin acetal homopolymer (polyoxymethylene POM), and the O rings are made from rubber.

The powder 212 is held within the receiving chamber by a paper filter 214 which serves as a retaining surround.

The receptacle 200 further comprises a cover for enclosing the powder within the receptacle, via an upper lid (216, 218) adapted to the sizes of the probes that will be used for the measurements.

In one embodiment, the lid is composed of a covering film 216 and a sealing lid 218, the whole being sealed with the main body 202 via a set of screws 220.

In one particular embodiment, the covering film is a plastic film with a thickness of between 50 μm and 200 μm.

In one particular embodiment, the sealing lid 218 is made from Delrin acetal homopolymer.

In one particular embodiment, the sealing lid 218 is sealed with the main body via a set of eight M6×12 mm polytetrafluoroethylene (PTFE) screws.

An air evacuation device 222 is provided at the level of the chamber containing the powder in order to suck out the air and avoid a reaction of the powder with the air. This makes it possible to ensure that the measurements of the powder are carried out without the influence of air.

In one embodiment, the air evacuation device is a tube attached to the main body 202 and in which a syringe (not shown) is placed in order to suck out the air.

In one particular embodiment, the tube is made from acrylic.

A person skilled in the art will be able to derive other particular embodiment forms from the general principles described. In particular, receptacles of different sizes may be designed, taking into account the principle that the materials used must not influence the measurements.

Thus, preferentially, the powder receptacles and their components can be made from a range of non-metallic materials: Delrin (polyoxymethylene POM), Plexiglas (polymethyl methacrylate), acrylic (polyacrylonitrile PAN), Mylar (PET polyester film), and rubber.

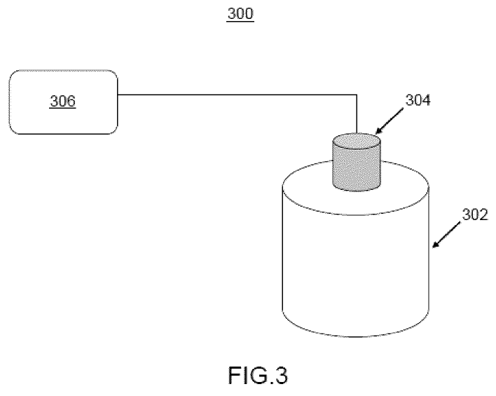

FIG. 3 is a schematic representation of the device for characterizing powder of the invention in one embodiment.

In general, the powder characterization device 300 of the invention is a simple and compact device. It is composed of a powder receptacle 302 calibrated to contain a powder sample the volume of which is representative of a powder recovered after an additive manufacturing operation.

The powder characterization device also comprises an eddy current (EC) emitting/receiving apparatus comprising an eddy current sensor 304 optimized for studying the powder.

The characterization device further comprises an acquisition and control system 306 coupled to the eddy current emitting/receiving apparatus and configured to determine impedance values from the signals received during the EC monitoring, and to determine the presence or absence of ferrite particles in the powder sample according to the measured impedance values.

Due to its compactness, the measurement device according to the invention can be used in the vicinity of an additive manufacturing machine or a powder recycling station.

Advantageously, due to its simple design and use, the measurement device of the invention enables the detection of the presence of ferrite in a volume of residual austenitic powders without requiring specific preparation of the sample to be characterized.

Another advantage of the device of the invention resides in the speed of characterization of the powder, achieved by recording of measurements by eddy currents at the surface of the powder contained in the receptacle and by immediate analysis of the measured impedance.

The eddy current (EC) technique is based on the detection of an electric signal emitted by an electrically conductive material subjected to an electromagnetic field that varies over time.

An eddy current sensor comprises a test head which generally includes at least one circuit having an emission function, which is supplied with AC current and allows a local electromagnetic field to be generated, and at least one receiver that is sensitive to this electromagnetic field.

The electromagnetic receiver often consists of a receiving coil (and optionally several connected together, for example differentially) across the terminals of which an electromotive force of the same frequency as that of the AC supply current is induced. The receiver may also be a Hall-effect sensor or else a magnetoresistive (MR) sensor. The latter family of sensors in particular contains anisotropic magnetoresistance (AMR) sensors, giant magnetoresistance (GMR) sensors, tunnel-effect magnetoresistance (TMR) sensors, and giant magnetoimpedance (GMI) sensors.

According to standard AFNOR NF EN 1330-5, October 1998, an eddy current transducer is a physical device including exciting elements and receiving elements. In the rest of the description, the term EC sensor designates such an eddy current transducer.

The arrangement and geometric shape of the emitting or receiving elements (emitting/receiving (E/R) elements) represent a “pattern”. A pattern may be made up of separate elements having emitting and receiving functions, of elements grouping together E/R functions, or of elements having one emitting function for a plurality of receivers.

According to various embodiments of the invention, the excitation and reception functions may be carried out in a differentiated manner in separate E/R mode or be carried out in combined E/R mode in order to measure the impedance response of the coil.

When the test head of an eddy current sensor is placed in the vicinity of a structure to be inspected or is moved over the surface of such a structure, the emitting circuit is supplied with a sinusoidal signal. An electromagnetic field of the same frequency is emitted into the structure to be inspected. Across the terminals of the receiving coil, an induced electromotive force results, this electromotive force being due, on the one hand, to the coupling between the emitting circuit and the receiving coil and, on the other hand, to the magnetic field radiated by the currents induced in the structure (eddy currents).

In uses for the detection of defects in structures, the circulation of the induced currents is altered in the event of the presence of a non-uniformity in the inspected material. The magnetic field receiver measures the magnetic field resulting from this alteration of the path of the induced currents.

In the context of the invention for the detection of ferrite particles, the circulation of the induced currents is altered depending on the impedance of the inspected material.

With ECs, the sensitivity of the measurement (or, in other words, the signal-to-noise ratio) increases as the distance between the emitting and receiving elements of the EC test head and the material to be inspected decreases. In addition, during the movement of the test head over the material, this distance (called the gap) must be as constant as possible in order to prevent bias from appearing in the measurements.

Since the EC sensor is a device that is quite sensitive to the gap, the inventors recommend working in contact with the surface of the powder, i.e., the head of the sensor comes into contact with the surface of the powder through the covering film 216.

To design the sensor, i.e. to design the coil, the principle is firstly to optimize the diameter of the coil in accordance with the dimensions of the receptacle and to set a range of working frequencies that is suitable for the material to be inspected.

Secondly, the number of layers and the number of turns have to be optimized in order to ensure that the resonant frequency of the coil is higher than the working frequency.

The EC sensor of the invention is thus optimized for the detection of the ferritic phase in the metal powder.

Specifically, it is necessary to use on the powder working frequencies that are higher than the working frequencies usually known for solid materials, since the electrical conductivity of powders is lower than that for solid materials.

Specifically to the context of the invention, which is focused on the detection of ferrite in austenitic materials, the working frequency range is within a range between 2 MHz and 10 MHz.

In one particular implementation, the working frequencies are taken within a range between 3 MHz and 7 MHz.

In another implementation, the working frequencies are taken within a range between 4 MHz and 5 MHz.

The inventors have determined that the ratio of the external diameter of the coil to the diameter of the receptacle should be between 0.2 and 0.35 in order to carry out global measurements over the surface of the receptacle while avoiding edge effects.

In one embodiment, the excitation frequency of the coil is chosen to be at least 1 MHz greater than the upper limit of the range of the working frequency, and preferentially 2 MHz greater.

In one particular embodiment, the sensor is composed of a coil without amplification in absolute mode (i.e. combined emission/reception). The diameter of the powder receptacle is 45 mm and the external diameter of the coil is 12 mm. The resonant frequency of the coil is 7.2 MHz and the impedance measurements for evaluating the presence of ferrite are carried out within a frequency range extending from 4 MHz to 5 MHz.

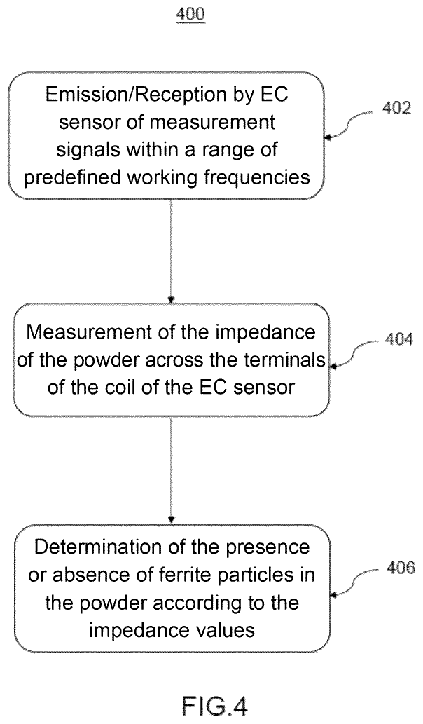

FIG. 4 illustrates the general steps of the method for characterizing a powder according to the invention.

The method is applied in particular to the determination of the presence of ferrite particles in a residual powder, in particular a 316 L-type austenitic powder.

Specifically, such a powder, after an operation of additive manufacturing according to a powder bed fusion process (PBF, L-PBF, EB-PBF), may contain particles that have resolidified in the form of ferrite. These ferritic particles may change the spreading quality of the powder prior to melting, which may give rise to the formation of defects in particular at the start of manufacture. It is therefore important to monitor the appearance of ferrite in the residual powder.

The general principle of the method 400 of the invention consists of quantifying the presence of ferrite particles in a powder sample representative of a residual powder remaining after an operation of additive manufacturing according to a powder bed fusion process. The determination of the proportion of ferrite particles is carried out according to the complex impedance value across the terminals of the receiving coil of an EC sensor operating in emission/reception mode (combined or separate E/R), within a range of working frequencies optimized for the powder being measured.

Specifically, in the presence of ferrite particles in the powder, the values for the conductivity and the permeability of the powder are altered, which will entail an alteration in the impedance (resistance and reactance), and which makes it possible to reveal the presence of ferrite.

The method of the invention is performed on a device having the general features of the device described with reference to FIG. 3, and having specific features determined according to the context of the additive manufacturing for which the testing of the powder is carried out.

Thus, the device on which the method of the invention is implemented brings together a powder receptacle the powder receiving chamber of which has been dimensioned beforehand and an EC sensor the working frequencies of which have been predefined.

A powder sample is placed in the receiving chamber, the powder is pressed towards the lid and the air that might be contained in the chamber is sucked out. The device is prepared.

In a first step 402, the method makes it possible to activate, via the EC sensor, the emission of electromagnetic measurement signals within a range of predefined working frequencies. The range of predefined frequencies is optimized for the type of powder contained in the receptacle and takes account of the requirement of not being too close to the resonant frequency of the coil of the EC sensor.

The head of the EC sensor is brought into the vicinity, through the covering film 216, of the surface of the powder contained in the receptacle for the entire duration of a sequence of measurements (a few seconds for recording around one hundred points). The information collected originates from a subsurface volume of the powder sample.

The covering film has a safety function since the material divided in powder form is potentially dangerous in and of itself. Such covering films have well-known characteristics. Moreover, the presence of this film also has an effect on the powder since it contributes to making the surface thereof more regular.

In a following step 404, the method makes it possible to measure the impedance across the terminals of the receiving coil (electromagnetic force) using an impedance meter, for each frequency of the range of predefined frequencies, according to defined measurement steps.

Then, in a following step 406, the method makes it possible to evaluate the quality of the remaining powder according to the impedance values measured for the powder sample.

Specifically, in the presence of a ferrite-free powder, for example a fresh powder, having values of conductivity σ1 and of permeability μ1, the impedance of the powder, denoted Z_{1powder},σ,μ₁, may be formulated according to the following equation:

$${Z}_{1 powder} , σ , {μ}_{1} = {R}_{powder} , σ , {μ}_{1} + {jX}_{powder} , σ , {μ}_{1} ,$$

where R_{powder} represents the real part and jX_{powder} the imaginary part of the impedance Z_{1powder},σ,μ₁.

If ferrite particles are present, the values of the conductivity and of the permeability of the powder are altered. They are respectively denoted σ₂ and μ₂. The impedance of the powder with ferrite is then formulated according to the following equation:

$${Z}_{2 powder} , σ , {μ}_{2} = {R}_{powder} , {σ}_{2} , {μ}_{2} + {jX}_{powder} , σ , {μ}_{2} .$$

Depending on the amount of ferrite present in the sample measured, the impedance values Z₁ and Z₂ may be consistent or different.

In one embodiment, step 406 of evaluating the quality of the remaining powder comprises a step of comparing the impedance measurements with predefined impedance values and of quantifying the presence of ferrite particles in the powder sample according to the differences between the measured values and the predefined values.

In one embodiment, step 406 of evaluating the quality of the remaining powder comprises a step of defining an acceptable ratio of ferrite particles present in the powder sample for retaining the remaining powder for a subsequent additive manufacture or not.

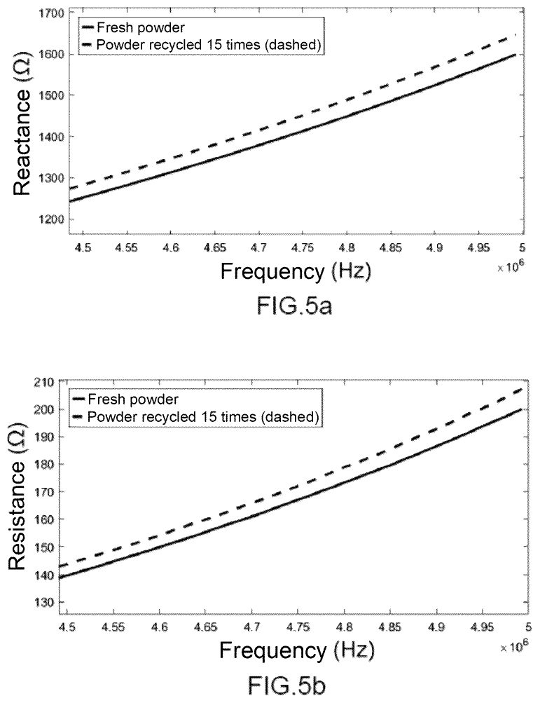

FIG. 5a and FIG. 5b respectively illustrate compared measurements of the reactance and of the resistance of a fresh 316 L steel powder (in solid lines) and of a 316 L steel powder that has been recycled 15 times (in dashed lines), measured within a range of frequencies from 4.5 MHz to 5 MHz in measurement steps of 5 kHz. The dashed curves illustrate that the recycled steel powder contains ferrite particles, since the values of reactance and resistance are different from the respective values for the fresh powder.

Thus, the method of the invention implemented on the described device makes it possible to determine, on the basis of measurements of impedance values for a powder sample representative of a residual powder, whether the powder can be reused for a subsequent additive manufacturing operation or not.

The impedance values measured on the sample are compared to impedance values for ferrite-free powder, for example a fresh or unrecycled powder. In the event of inconsistency between the values, and depending on a level of contamination with the ferrite particles which may be predefined as being an acceptable threshold, the powder from which the sample was taken can be used or reused for a subsequent additive manufacturing operation.

1. A device for characterizing a residual powder remaining after an operation of additive manufacturing by a powder bed fusion process, the device comprising:
a powder receptacle for receiving a sample of said residual powder, the receptacle being calibrated in terms of its dimensions to contain said sample and comprising an upper lid composed of a covering film and a sealing lid;
an eddy current emitting/receiving apparatus (304), having a coil with a diameter dimensioned in accordance with the dimensions of the powder receptacle, and configured to emit to the surface of the powder sample, through the covering film, electromagnetic signals within a range of predefined working frequencies; and
an acquisition and control system (306), configured to:
measure, for each working frequency, impedance values across the terminals of a receiving coil of the eddy current emitting/receiving apparatus; and
evaluate the quality of the remaining powder according to the impedance values measured for the powder sample.

2. The device according to claim 1, wherein the powder receptacle comprises a main body comprising at least one powder receiving chamber, and a bearing block for pressing the powder, the powder receiving chamber being dimensioned to receive a powder sample representative of the remaining powder.

3. The device according to claim 1, wherein the powder receptacle comprises an air evacuation device configured to suck out the air contained in the powder receptacle.

4. The device according to claim 1, wherein the receptacle is made from a range of non-metallic materials, including in particular Delrin (polyoxymethylene POM), Plexiglas (polymethyl methacrylate), acrylic (polyacrylonitrile PAN), Mylar (PET polyester film), rubber.

5. The device according to claim 1, wherein the eddy current emitting/receiving apparatus comprises a sensor composed of a coil without amplification in absolute mode, the diameter of the coil being dimensioned in accordance with the dimensions of the powder receptacle.

6. The device according to claim 1, wherein the eddy current emitting/receiving apparatus comprises a sensor composed of a coil of which the number of layers and the number of turns is predefined in order to ensure that the resonant frequency of the coil is higher than the range of working frequencies of the sensor.

7. The device according to claim 1, wherein the acquisition and control system comprises an impedance meter for measuring the impedance values.

8. A method for characterizing a residual powder remaining after an operation of additive manufacturing by a powder bed fusion process, the method being implemented for a sample of said residual powder contained in a powder receptacle calibrated in terms of its dimensions to contain said sample and comprising an upper lid composed of a covering film and a sealing lid, and comprising the steps of:
(402) activating, via an eddy current EC sensor having a coil with a diameter dimensioned in accordance with the dimensions of the powder receptacle, the emission of electromagnetic signals, within a range of predefined working frequencies, to the surface of said powder sample through the covering film;
(404) measuring, for each working frequency, the impedance across the terminals of the receiving coil of the EC sensor; and
(406) evaluating the quality of the remaining powder according to the impedance values measured for the powder sample.

9. The method according to claim 8, wherein the activation step of the EC sensor is carried out within a range of working frequencies between 2 MHz and 10 MHz.

10. The method according to claim 8, wherein the activation step of the EC sensor is carried out within a range of working frequencies between 4 MHz and 5 MHz.

11. The method according to claim 8, wherein step 406 of evaluating the quality of the remaining powder comprises a step of comparing the impedance measurements with predefined impedance values and of quantifying the presence of ferrite particles in the powder sample according to the differences between the measured values and the predefined values.

12. The method according to claim 8, wherein step 406 of evaluating the quality of the remaining powder comprises a step of defining an acceptable ratio of ferrite particles present in the powder sample for retaining the remaining powder for a subsequent additive manufacture or not.

13. The method according to claim 8, wherein the characterization of a powder consists of characterizing a sample of 316 L-type austenitic powder.
